(19) United States
(12) Patent Application Publication
(10) Pub. No.: US 2023/0326017 A1
(43) Pub. Date: Oct. 12, 2023

(54) SYSTEM AND METHOD FOR AUTOMATICALLY MEASURING SPINAL PARAMETERS

(71) Applicants: AJOU UNIVERSITY INDUSTRY-ACADEMIC COOPERATION FOUNDATION, Suwon-si (KR); UIF (UNIVERSITY INDUSTRY FOUNDATION), YONSEI UNIVERSITY, Seoul (KR)

(72) Inventors: Sunghyeon NOH, Seoul (KR); Yoon HA, Seoul (KR)

(73) Assignees: AJOU UNIVERSITY INDUSTRY-ACADEMIC COOPERATION FOUNDATION, Suwon-si (KR); UIF (UNIVERSITY INDUSTRY FOUNDATION), YONSEI UNIVERSITY, Seoul (KR)

(21) Appl. No.: 18/124,902

(22) Filed: Mar. 22, 2023

(30) Foreign Application Priority Data
Mar. 22, 2022 (KR) 10-2022-0035147

(51) Int. Cl. A61B 5/00; G06T 7/60; G06T 7/00

(52) U.S. Cl. CPC G06T 7/0012; A61B 5/4566; A61B 5/7267; G06T 7/60; G06T 2207/20081; G06T 2207/30012

(57) ABSTRACT

Provided are a system and method of automatically measuring spinal parameters. The system includes an image acquisitor configured to acquire an overall image of a spine and a parameter extractor configured to generate a learning model for extracting designated points from various spinal images and detecting relationships between the extracted points as angles, search for the designated points in the overall spinal image of the image acquisitor using the generated learning model, and check angular relationships between the points.

### CROSS-REFERENCE TO RELATED APPLICATION

This application claims priority to and the benefit of Korean Patent Application No. 10-2022-0035147, filed on Mar. 22, 2022, the disclosure of which is incorporated herein by reference in its entirety.

### BACKGROUND

### 1. Field of the Invention

The present invention relates to a system and method for automatically measuring spinal parameters, and more particularly, to a system and method for measuring spinal parameters using artificial intelligence (AI)-based point detection.

### 2. Discussion of Related Art

In general, a medical imaging apparatus is equipment for acquiring an image of an internal structure of a target object. Such a medical imaging apparatus is a non-invasive examination device that is used without causing pain to the human body, and in which structural details within the body, internal tissues, flows of fluids, etc. are imaged, processed, and then shown to a medical worker. Medical workers may diagnose health conditions and diseases of patients using medical images output from medical imaging apparatuses.

Medical imaging apparatuses include magnetic resonance imaging (MRI) equipment for providing magnetic resonance images, computed tomography (CT) equipment, X-ray equipment, ultrasound diagnostic equipment, etc. Medical images acquired from such medical imaging apparatuses may be used for diagnosing diseases.

Meanwhile, in diagnosis based on medical images, a measurement process may be very important for diagnosing various lesions and also determining the stage of disease progression. Here, reference points, such as anatomical landmarks, may be important for accurate and highly reproducible measurement, particularly in measurement based on two-dimensional (2D) planar medical images. However, anatomical landmarks may be present at different positions in different subjects to be diagnosed, and may be determined differently according to external factors such as the posture of the subject and the skill level of the medical worker. For this reason, measurement based on medical images, particularly, measurement based on 2D planar medical images, may show low accuracy and reproducibility. Here, inaccurate measurement values may degrade the reliability of the diagnosis of a disease and the evaluation results of disease progression.

Particularly, spinal diseases, such as scoliosis, may be diagnosed by measuring the Cobb angle which is an angle of curvature through CT, MRI, and X-ray imaging. However, inter-observer and intra-observer reproducibility of measurement is low because there is a high possibility that the subjectivity of the medical worker making the diagnosis is involved. Due to the low reproducibility, scoliosis is determined to have worsened when the Cobb angle increases by 5° or more. However, when the reproducibility is ensured, it is possible to interpret a smaller increase than that as progression of the disease. Therefore, ensuring reproducibility through explainable automation is very important.

Korean Patent Publication No. 10-2021-0127849 (“System and method for determining severity of ankylosing spondylitis using spinal imaging based on artificial intelligence,” published on Oct. 25, 2021) discloses a configuration for generating a trained learning model by training a learning model with scores of corners in spine images, checking the score of each corner in an image using the trained model, and adding up the scores.

However, such a method according to the related art has a disadvantage in that it is difficult to analyze the overall shape of the spine and determine the cause of a spinal disease accordingly.

### SUMMARY OF THE INVENTION

The present invention is directed to providing a system and method for automatically measuring spinal parameters which enable a user to check the overall shape of a spine.

The present invention is also directed to providing a system and method for automatically measuring spinal parameters which enable a user to check an accurate spinal condition by setting various observation points and detecting a relative angle of each point.

According to an aspect of the present invention, there is provided a system for automatically measuring spinal parameters, the system including an image acquisitor configured to acquire an overall image of a spine and a parameter extractor configured to generate a learning model for extracting designated points from various spinal images and detecting relationships between the extracted points as angles, search for the designated points in the overall spinal image of the image acquisitor using the generated learning model, and check angular relationships between the points.

The image acquisitor may be provided as at least one image acquisitor, and the image acquisitor may provide a captured image through a network to the parameter extractor.

The parameter extractor may measure pelvic incidence, pelvic tilt, sacral slope, lumbar lordosis, L4S1 lordosis, thoracic kyphosis, and T1 pelvic angles using the angular relationships between the extracted points.

According to another aspect of the present invention, there is provided a method of automatically measuring spinal parameters by a computing device including a processor, the method including operation a) classifying various spinal images into training data and test data and performing training with the training data to generate a learning model, operation b) extracting designated points from an input overall spinal image, and operation c) extracting parameters using angular relationships between the points.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of a system for automatically measuring spinal parameters according to an exemplary embodiment of the present invention;

FIG. 2 is an example view of points extracted according to the present invention; and

FIGS. 3 to 7 are example views of parameter extraction according to the present invention.

### DETAILED DESCRIPTION OF EXEMPLARY EMBODIMENTS

Hereinafter, a system and method for automatically measuring spinal parameters according to exemplary embodiments of the present invention will be described in detail with reference to the accompanying drawings.

Exemplary embodiments of the present invention are provided to describe the present invention more completely to those of ordinary skill in the art. The exemplary embodiments disclosed herein may be modified into several other forms, and the present invention is not limited thereto. Rather, these embodiments are provided to make the present invention through and complete and fully convey the spirit of the present invention to those of the ordinary skill in the art.

Terminology used in this specification is for the purpose of describing exemplary embodiments and is not intended to limit the present invention. As used herein, a singular expression may include a plural expression unless the context clearly indicates to the contrary. As used herein, the terms “comprise” and/or “comprising” specify the existence of stated shapes, numerals, steps, operations, members, elements, and/or a group thereof and do not exclude the existence or addition of one or more other shapes, numerals, steps, operations, members, elements, and/or a group thereof. As used herein, the term “and/or” includes any and all combinations of one or more associated listed items.

In this specification, terms such as “first,” “second,” etc. may be used to describe various members, areas, and/or parts, but the members, areas, and or parts are not limited by the terms. Also, the terms do not denote a specific order, vertical relationship, or superiority and are only used for the purpose of distinguishing one member, area, or part from another member, area, or part. Therefore, a first member, area, or part to be described below may be named a second member, area, or part without departing from the present invention.

The exemplary embodiments of the present invention will be described below with reference to the drawings which schematically show the exemplary embodiments of the present invention. In the drawings, modifications of shown shapes may be expected depending on, for example, the manufacturing technology and/or tolerance. Therefore, embodiments of the present invention should not be interpreted as being limited to specific shapes of areas illustrated herein and should include, for example, changes in the shapes caused by manufacturing.

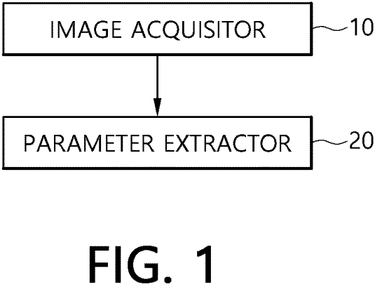

FIG. 1 is a block diagram of a system for automatically measuring spinal parameters according to an exemplary embodiment of the present invention.

Referring to FIG. 1, the present invention includes an image acquisitor 10 which acquires an overall X-ray image of a spine and a parameter extractor 20 which generates a learning model for learning designated points from various spinal images and detecting relationships between the designated points as angles, searches for the designated points in the overall spinal image of the image acquisitor 10 using the generated learning model, and checks angular relationships between the points.

Operations of the system of the present invention configured as described above will be described in further detail below.

First, the image acquisitor 10 is a device which images an X-ray image of a patient. There may be a plurality of image acquisitors 10 as necessary, and images captured by the plurality of image acquisitors 10 may be provided to the parameter extractor 20.

Image data may be provided as the images from the image acquisitors 10 to the parameter extractor 20 through a network.

The network may be a wired or wireless network.

The wired or wireless network may be a local network, such as an intrahospital network or the like, or a wide area network with no areal limitation.

As a network configuration, any known data transmission network may be used.

The parameter extractor 20 includes a network adapter for receiving image data from the image acquisitor 10 and a storage device for storing the image data.

The parameter extractor 20 may include a processor which generates and executes the learning model for detecting designated positions (points) in a spinal image and detecting relationships between the points as angles, and a display which displays detection results.

In other words, the parameter extractor 20 may be a computing device such as a computer or the like.

The parameter extractor 20 may employ a U-Net which is an end-to-end fully convolutional network proposed for the purpose of image processing (particularly, segmentation) in the biomedical field.

To generate a learning model using the U-Net, 1,000 pieces of training data and 200 pieces of test data are used in the present invention. To this end, 1,200 spinal images are used in total.

Point detection is an operation of detecting major corner coordinates of a spine according to results obtained by learning spinal images. The parameter extractor 20 detects points according to the learning model using differences with surrounding pixels and the like.

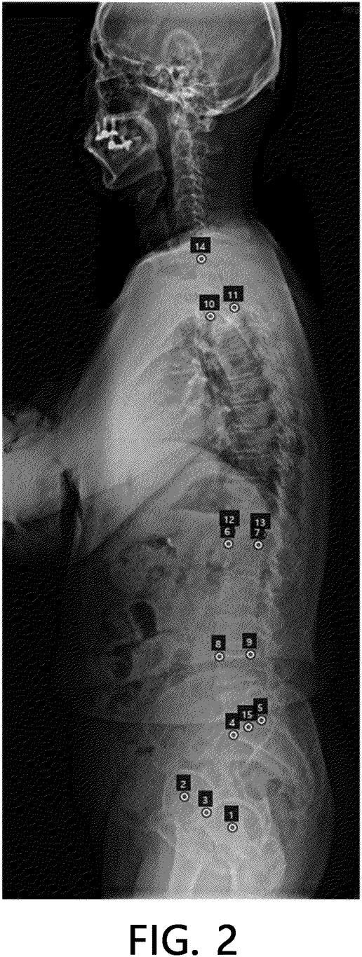

FIG. 2 is an example view of points detected by the parameter extractor 20.

Referring to FIG. 2, the parameter extractor 20 detects 15 points.

Each of the 15 points may be displayed with a given number. 1 and 2 indicate femur head centers, and 3 indicates the center point of 1 and 2.

4 is an S1 endplate anterior point, 5 is an S1 endplate posterior point, 6 is an L1 upper endplate anterior point, and 7 is an L1 upper endplate posterior point.

8 is an L4 upper endplate anterior point, and 9 is an L4 upper endplate posterior point.

10 is a T4 upper endplate anterior point, and 11 is a T4 upper endplate posterior point.

Also, 12 is a T12 lower endplate anterior point, 13 is a T12 lower endplate posterior point, 14 is a T1 vertebra center point, and 15 is an S1 endplate center point.

The signs S1, L1, T4, etc. are, as is known, symbols according to the position in the spine.

S1 indicates the first sacral vertebra, L1 indicates the first lumbar vertebra, L4 indicates the fourth lumbar vertebra, T4 indicates the fourth thoracic vertebra, and T12 indicates the twelfth thoracic vertebra.

The parameter extractor 20 extracts various parameters using the extracted 15 points.

As a result of evaluating the above-described learning model generation, it is found that each point may have an error distance.

Results of checking error distances are shown in Table 1.

TABLE 1

| Point | Error distance (mm) |
|---|---|
| 1 | 3.8333±1.0521 |
| 2 | 4.6051±1.6098 |
| 3 | 3.9715±1.7714 |
| 4 | 2.9460±1.3565 |
| 5 | 3.7371±1.8314 |
| 6 | 4.7477±2.8213 |
| 7 | 3.6955±0.6394 |
| 8 | 2.2602±2.6862 |
| 9 | 3.6700±3.5009 |
| 10 | 2.8028±2.4216 |
| 11 | 3.7659±2.6522 |
| 12 | 2.2667±2.1256 |
| 13 | 2.3415±2.6610 |
| 14 | 3.3657±2.5721 |
| 15 | 2.6951±1.8345 |
| mean | 4.5715±1.5248 |

Error distances in the above table may be reduced according to the amount of training data and the number of times that the training is performed.

In this case, the previously detected points may be means for extracting pelvic incidence, pelvic tilt, sacral slope, lumbar lordosis, L4A1 lordosis, thoracic kyphosis, and T1 pelvic angles.

This will be described in further detail below.

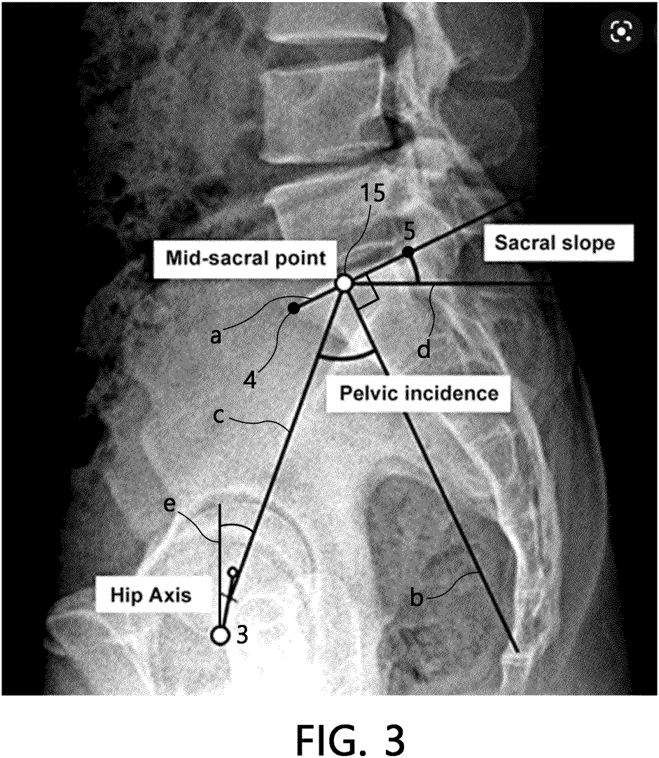

FIG. 3 is a view illustrating an example of extracting parameters of pelvic incidence, a pelvic tilt, and a sacral slope.

The parameter extractor 20 detects points and then determines a line segment c that connects the points 3 and 15.

Also, the parameter extractor 20 determines a virtual line segment b orthogonal to a line segment a passing through the points 4, 15, and 5 and then extracts an angular parameter of pelvic incidence by detecting the angle between the line segments b and c.

There is a normal range of pelvic incidence angle. When a pelvic incidence angle deviates from the normal range, this may be determined to be abnormal.

The detected angle may have an error angle according to the error distances defined in Table 1 above. Examples of error angles are shown in Table 2 below.

TABLE 2

| parameter | Error angle (°) |
|---|---|
| Pelvic incidence | 2.6868±0.0124 |
| Pelvic tilt | 0.9429±0.4012 |
| Sacral slope | 2.4324±1.3412 |
| Lumbar lordosis | 4.8103±1.7851 |
| L4S1 lordosis | 3.2329±0.9487 |
| Thoracic kyphosis | 5.7665±1.8781 |
| T1 pelvic angle | 0.8834±0.2124 |

Such error angles may be further reduced with accumulation of data and an increase in the number of times that the training is performed. Therefore, according to the present invention, it is possible to detect a more accurate angle than in a method according to the related art.

Referring back to FIG. 3, a pelvic tilt angle may be detected by finding the angle between a line segment e vertically passing through the point 3 and the line segment c.

Also, an acute angle between the line segment a and a line segment d is a sacral slope.

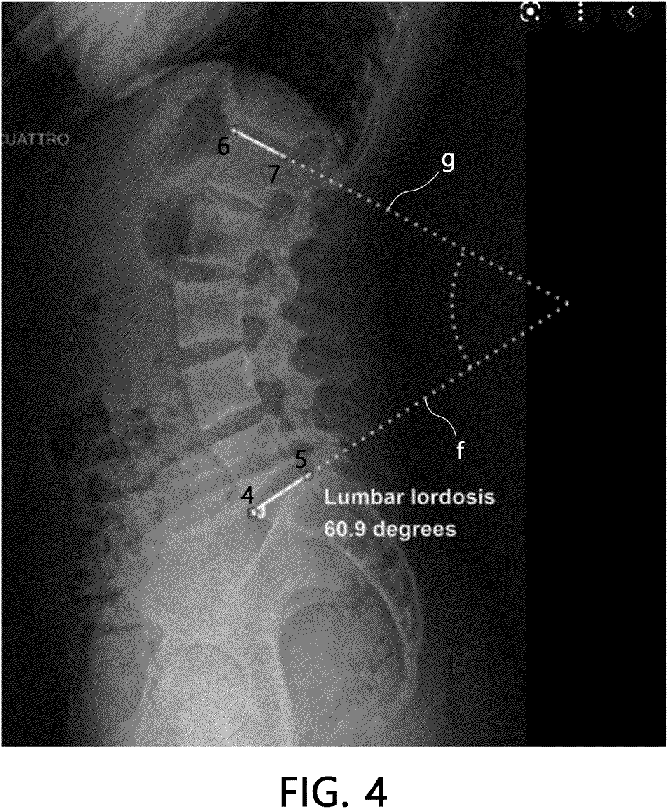

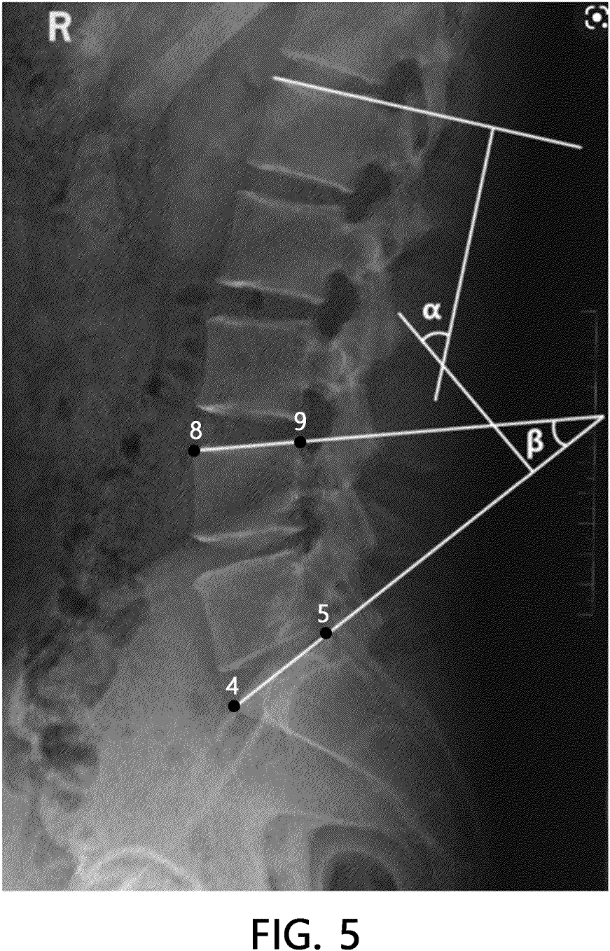

After that, as shown in FIG. 4, an acute angle between an extension f of a line segment connecting the points 4 and 5 and an extension g of a line segment connecting the points 6 and 7 is a lumbar lordosis angle.

Referring to FIG. 4, an acute angle β between an extension f of the line segment connecting the points 4 and 5 and an extension f of a line segment connecting the points 8 and 9 is an L4S1 lordosis angle.

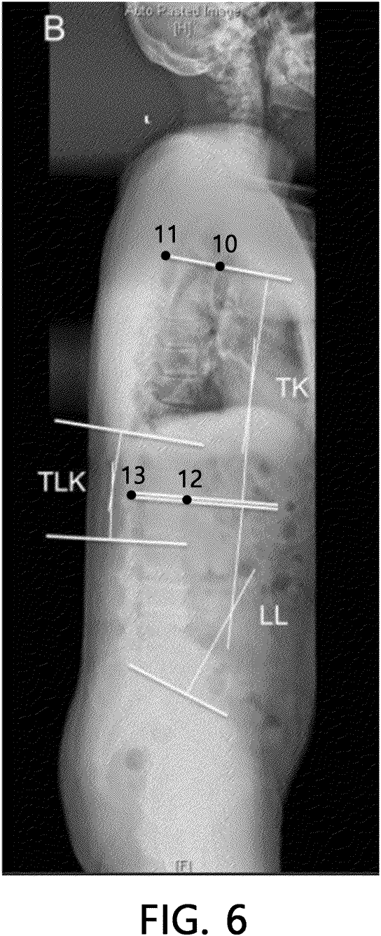

Also, referring to FIG. 6, a thoracic kyphosis angle may be detected by finding an angle between an extension of a line segment connecting the points 11 and 10 and an extension of a line segment connecting the points 13 and 12.

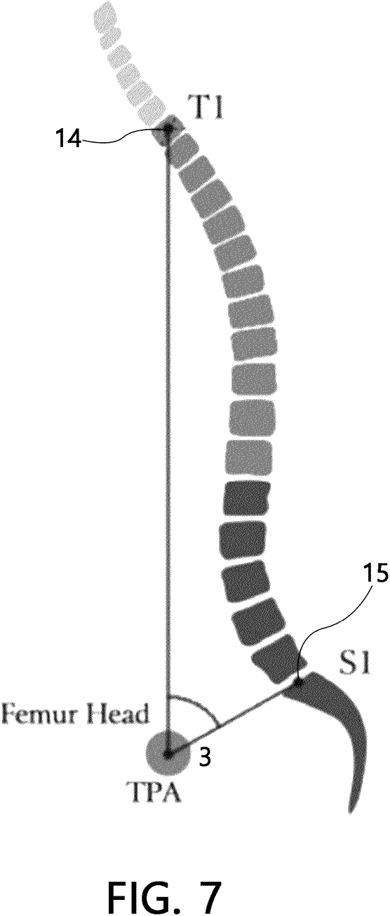

Finally, referring to FIG. 7, an angle between the points 14 and 15 with respect to the point 3 is detected as a T1 pelvis angle.

As described above, with the present invention, it is possible to learn images, detect points having specific location coordinates, and extract accurate parameters using angular relationships between the points.

According to the present invention, it is possible to detect specific points of a spine in an overall spinal image using AI, check an accurate overall spine shape using relationships between the points, and make a diagnosis.

It is apparent to those of ordinary skill in the art that the present invention is not limited to the above embodiments and can be implemented in various modified or altered forms without departing from the technical scope of the present invention.

What is claimed is:

1. A system for automatically measuring spinal parameters, the system comprising:
an image acquisitor configured to acquire an overall image of a spine; and
a parameter extractor configured to generate a learning model for extracting designated points from various spinal images and detecting relationships between the extracted points as angles, search for the designated points in the overall spinal image of the image acquisitor using the generated learning model, and check angular relationships between the points.

2. The system of claim 1, wherein the image acquisitor is provided as at least one image acquisitor, and
the image acquisitor provides a captured image through a network to the parameter extractor.

3. The system of claim 2, wherein the parameter extractor measures pelvic incidence, pelvic tilt, sacral slope, lumbar lordosis, L4S 1 lordosis, thoracic kyphosis, and T1 pelvic angles using the angular relationships between the extracted points.

4. A method of automatically measuring spinal parameters by a computing device including a processor, the method comprising:
operation a) classifying various spinal images into training data and test data and performing training with the training data to generate a learning model;
operation b) extracting designated points from an input overall spinal image; and
operation c) extracting parameters using angular relationships between the points.

5. The method of claim 4, wherein the points extracted in operation b) are an S1 endplate anterior point, an S1 endplate posterior point, an L1 upper endplate anterior point, an L1 upper endplate posterior point, an L4 upper endplate anterior point, an L4 upper endplate posterior point, a T4 upper endplate anterior point, a T4 upper endplate posterior point, a T12 lower endplate anterior point, a T12 lower endplate posterior point, a T1 vertebra center point, and an S1 endplate center point.

6. The method of claim 4, wherein the parameters include pelvic incidence, pelvic tilt, sacral slope, lumbar lordosis, L4S1 lordosis, thoracic kyphosis, and T1 pelvic angles which are detected using the angular relationships between the extracted points.
